(19) United States
(12) Patent Application Publication
(10) Pub. No.: US 2019/0345432 A1
(43) Pub. Date: Nov. 14, 2019

(54) CULTURE EQUIPMENT OF MICROORGANISMS AND METHOD FOR COUNTING NUMBER OF MICROORGANISMS USING SAME

(71) Applicant: JNC CORPORATION, Tokyo (JP)

(72) Inventor: Hajime TERAMURA, Kanagawa (JP)

(73) Assignee: JNC CORPORATION, Tokyo (JP)

(21) Appl. No.: 16/462,544

(22) PCT Filed: Oct. 12, 2017
(86) PCT No.: PCT/JP2017/036991

§ 371 Date: May 21, 2019

(30) Foreign Application Priority Data
Nov. 28, 2016 (JP) 2016-229753

(51) Int. Cl. C12M 1/12; C12N 1/20

(52) U.S. Cl. CPC C12M 25/16; C12N 1/20

(57) ABSTRACT

A culture equipment in which culture equipment can be easily produced with high handleability, and the number of the microorganisms in the analyte can be easily counted. The culture equipment of microorganisms has (a) an upper member, (b) a lower member having a recess, and (c) a medium ingredient, and (c) the medium ingredient contains (c1) at least one selected from polyacrylic acid and/or a salt thereof and a derivative thereof, and (c2) a nutritional ingredient. Then, (a) the upper member preferably has a projection having a shape that can be mutually fitted to the recess of (b) the lower member through (c) the medium ingredient.

### TECHNICAL FIELD

The invention relates to culture equipment for simply counting the number of microorganisms in an analyte.

### BACKGROUND ART

As a method for counting the number of microorganisms, a pour plate method, an agar spread plate method and the like are known (Non-Patent literature No. 1).

An agar medium used for culturing the microorganisms in the methods described above is a material prepared by solidifying a medium in which a nutritional ingredient and a selective ingredient are dissolved thereinto with agar, and needs to be pre-prepared prior to culturing and counting the microorganisms. Moreover, in the agar spread plate method, upon applying an analyte onto a plate medium, the analyte is applied onto the medium while the analyte is completely absorbed into the medium, and therefore the method also has a problem of needing time for operation.

In recent years, development has been made in various manners on dry simple culture equipment in which pre-preparation of the medium is unnecessary in order to detect and count the microorganisms in a simpler and further efficient manner. In such culture equipment, if a liquid analyte is added thereto when in use, the medium is formed by a moisture content thereof, and can be directly provided for culture.

For example, Patent literature No. 1 discloses a sheet-shaped culturing apparatus provided with an adhesive layer, a chilled water-soluble gelling agent powder layer containing a nutritional ingredient, and a cover sheet, on a top surface part of a water-proof base. Patent literature No. 2 discloses a simple medium having a water-soluble gelling agent and a fibrous absorbent sheet having a mesh, on a top surface of a water-proof base. Patent literature No. 3 discloses sheet-shaped culture equipment in which an absorbent polymer layer and a porous matrix layer are sequentially laminated on a top surface of a water-proof base. Patent literature No. 4 discloses sheet-shaped culture equipment in which a frame on which an analyte spreads is provided on a base sheet, and a medium solution containing an adhesive ingredient and a gelling agent is subjected to patter formation within the frame. Such a frame is composed of a hydrophobic resin having a contact angle set to a specific value, and a sample solution spreads only within the frame.

### CITATION LIST

### Patent Literature

Patent literature No. 1: JP H2-49705 B.

Patent literature No. 2: JP 2000-325072 A.

Patent literature No. 3: WO 97/24432 A.

Patent literature No. 4: JP 2015-204845 A.

### Non-Patent Literature

Non-Patent literature No. 1: Manual to Practice Bacteriology, 2nd, p59, 4.3 Bacteria counting and culture method, edited by The Institute of Medical Science, The University of Tokyo, Maruzen Co., Ltd.

### SUMMARY OF INVENTION

### Technical Problem

In the culture equipment described in Patent literature No. 1, an analyte is applied to a space between a water-proof base and a top surface film to perform operation of pressing the resulting set from above the top surface film using a tool called a spreader to spread a sample solution in a predetermined area. The operation requires a flat surface, and unless the operation is performed cautiously, a sample is liable to flow out to a surrounding, far from homogeneously spreading of the sample, and the culture equipment has a problem on handleability.

In the culture equipment described in Patent literature No. 2 and No. 3, a porous matrix, including a nonwoven fabric, is used, and therefore a liquid analyte added thereto is diffused by a capillary phenomenon to homogeneously spread wholly on the equipment, and therefore handleability is high. However, as a matter of convenience of using the porous matrix, upon production thereof, the art has difficulty in controlling a dry state of a solvent (Patent literature No. 2), and a procedure of laminating the porous matrix layer and a medium layer is complicated (Patent literature No. 3). Moreover, the art also has a drawback in which colonies after culture become hard to observe by irregular reflection caused on a medium surface by irregularity on a surface of the porous matrix or opacity of the porous matrix itself.

In the culture equipment described in Patent literature No. 4, no porous matrix is used, but a flat place or cautiousness is required upon the operation so as to prevent the analyte from being overflowed from the frame serving as a dike when the liquid analyte spreads. Moreover, the frame is formed of a material set to a specific contact angle, but depending on a kind of the analyte, for example, when food and drink or the like containing oil, protein or the like is applied as the analyte, water repellency of the frame is assumed to change, and the art may be reasonably referred to as having a problem of versatility as the culture equipment.

In view of such a situation, an object of the invention is to provide culture equipment of microorganisms, in which the culture equipment has high handleability, and can be simply produced, and the number of microorganisms in the analyte can be easily counted.

### Solution to Problem

The present inventors have diligently continued to conduct study in order to solve the problems as described above. As a result, the present inventors have arrived at a conception in which, if a medium ingredient is provided in a space surrounded by a dish-shaped member having a recess and a lid member having a projection upon fitting both members, an analyte can be homogeneously diffused upon application thereof without using a tool or a capillary phenomenon, and a medium can be formed in a significantly short period of time, and therefore culture and counting of microorganisms can be realized by simple operation. Then, the present inventors have found that polyacrylic acid and/or a salt thereof is preferred as a gelling agent forming the medium from a viewpoint of simplicity of the operation and high visibility from outside, and have completed the invention.

More specifically, the invention includes the items described below.

Item 1. Culture equipment of microorganisms, including (a) an upper member; (b) a lower member having a recess; and (c) a medium ingredient, wherein (c) the medium ingredient contains (c1) a polymer compound that can form non-flowable transparent gel without passing through dissolution by heating and without depending on cooling, and (c2) a nutritional ingredient.

Item 2. The culture equipment according to item 1, wherein (c1) the polymer compound can be hydrated in an amount 10 times or more its own weight.

Item 3. The culture equipment according to item 1 or 2, wherein the gel causes no syneresis.

Item 4. The culture equipment according to any one of items 1 to 3, wherein (c1) the polymer compound has acrylic acid as a monomer unit.

Item 5. The culture equipment according to item 4, wherein (c1) the polymer compound is at least one selected from polyacrylic acid and/or a salt thereof and a derivative thereof.

Item 6. The culture equipment according to any one of items 1 to 5, wherein (a) the upper member has a projection having a shape that can be mutually fitted to the recess of (b) the lower member through (c) the medium ingredient.

Item 7. The culture equipment according to item 6, wherein (c) the medium ingredient is coated onto at least a part of the projection of (a) the upper member and/or the recess of (b) the lower member.

Item 8. The culture equipment according to any one of items 1 to 7, wherein (a) the upper member and/or (b) the lower member is transparent.

Item 9. A method, in which microorganisms in an analyte are cultured and the number of the microorganisms is counted by using the culture equipment according to any one of items 1 to 8.

Item 10. The method according to item 9, including:

a step of adding an analyte to a recess of (b) a lower member;

a step of fitting a projection of (a) an upper member to the recess of (b) the lower member;

a step of culturing microorganisms contained in the analyte; and

a step of counting the number of colonies of the microorganisms.

Item 11. The method according to item 9, wherein (a) an upper member of culture equipment includes a projection having a shape that can be mutually fitted to a recess of (b) a lower member through (c) a medium ingredient, including:

a step of adding an analyte to the recess of (b) the lower member;

a step of fitting the projection of (a) the upper member to the recess of (b) the lower member;

a step of culturing microorganisms contained in the analyte; and

a step of counting the number of colonies of the microorganisms.

In addition, the analyte herein is not particularly limited, but is ordinarily a liquid analyte, and specific examples thereof include an aqueous liquid analyte such as drinking water, soft drink, water for industrial use, water for pharmaceutical use, dialysis water and urine.

Moreover, the microorganisms herein ordinarily refer to Coliform bacteria, Staphylococcus species bacteria, Vibrio species bacteria, Enterococcus species bacteria, Bacillus species bacteria, Yeasts, molds and the like.

### Advantageous Effects of Invention

According to the invention, microorganisms in an analyte can be cultured by simple operation, and the number thereof can be easily counted. Moreover, culture equipment according to the invention does not have a complicated configuration, and therefore can be easily produced.

### BRIEF DESCRIPTION OF DRAWINGS

FIG. 1 is a diagram showing one aspect of culture equipment according to the invention, in which (A) shows an orthographic diagram of the culture equipment in a state in which an upper member and a lower member are not stacked, (B) shows one example of a cross sectional view taken along line A-A′ in (A), (B′) shows another example of the cross sectional view taken along line A-A′ in (A), (C) shows one example of a cross sectional view taken along line B-B′ in (A), and (C′) shows another example of the cross sectional view taken along line B-B′ in (A).

FIG. 2 is a diagram showing one aspect of culture equipment according to the invention, in which (A) shows an orthographic diagram of the culture equipment in a state in which an upper member is not fitted to a lower member, (B) shows one example of a cross sectional view taken along line A-A′ in (A), (B′) shows another example of the cross sectional view taken along line A-A′ in (A), (C) shows one example of a cross sectional view taken along line B-B′ in (A), and (C′) shows another example of the cross sectional view taken along line B-B′ in (A).

FIG. 3 is a diagram showing one aspect of culture equipment according to the invention, in which (A) shows an orthographic diagram of the culture equipment in a state in which an upper member is fitted to a lower member, (B) shows one example of a cross sectional view taken along line C-C′ in (A), and (C) shows another example of the cross sectional view taken along line C-C′ in (A).

FIG. 4 is an oblique projection diagram showing a use aspect example of culture equipment according to the invention.

FIG. 5 shows a photograph of red colonies detected in culture equipment in Example.

### DESCRIPTION OF EMBODIMENTS

Culture equipment according to the invention will be described with reference to drawings.

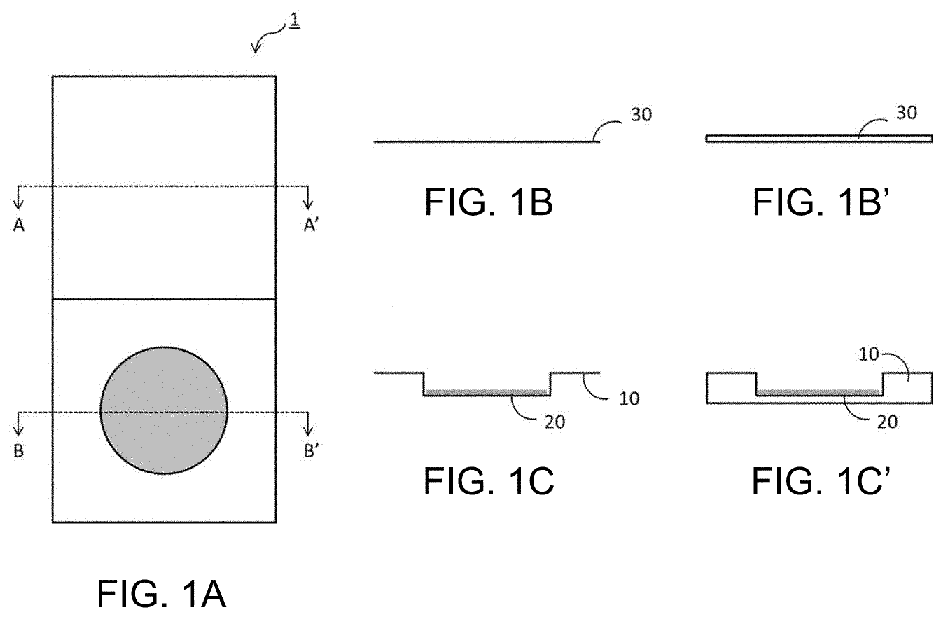

The culture equipment according to the invention has (a) upper member (30), (b) lower member (10) having a recess, and (c) medium ingredient (20) (FIG. 1). The culture equipment is ordinarily used by putting upper member (30) on the recess of lower member (10) in a manner of covering the recess of lower member (10). In a state of putting upper member (30) thereon, a proper space exists between the upper member and the recess of the lower member, and the medium ingredient exists therein. In such a state, a space surrounded by the upper member, and a bottom surface and a side surface of the recess of the lower member serves as the space in which a medium formed of the medium ingredient and an analyte exists (hereinafter, also described as “medium region”).

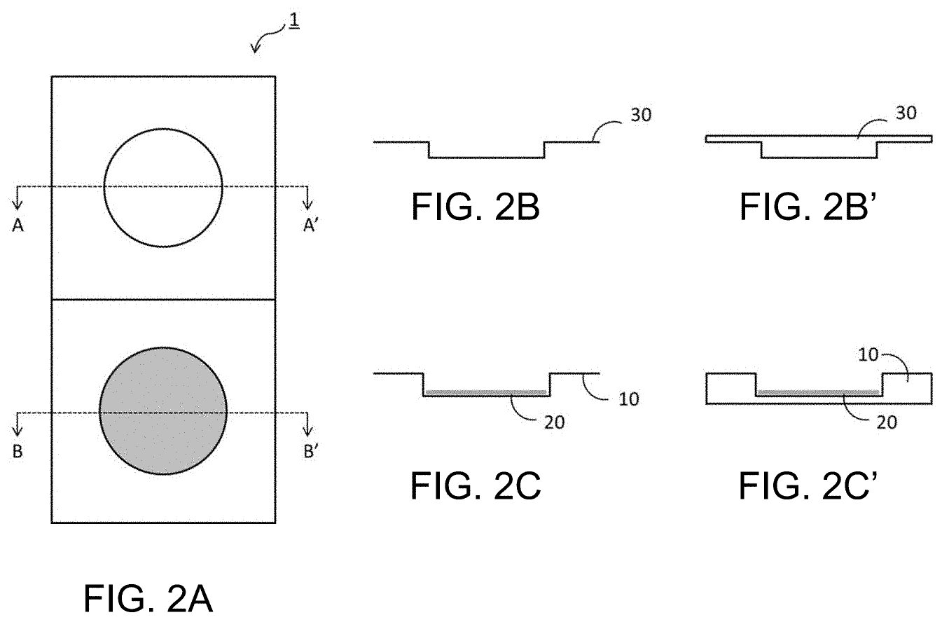

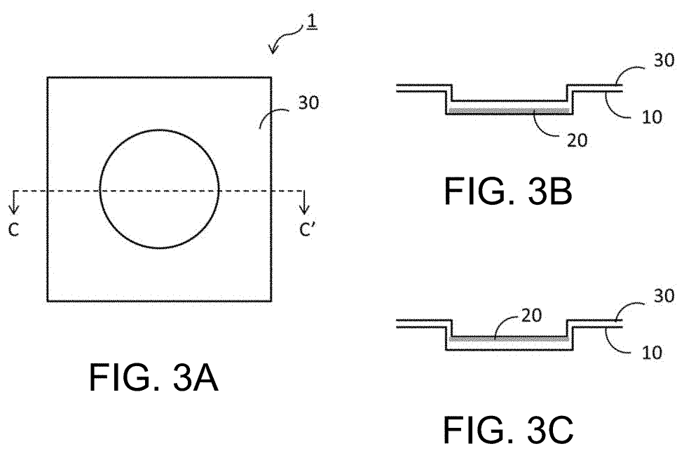

In a preferred aspect, upper member (30) has a projection having a shape that can be mutually fitted to the recess of lower member (10) through the medium ingredient (FIG. 2). In the aspect described above, for example, as shown in FIG. 3, a columnar projection of the upper member is fitted to a columnar recess of the lower member, the recess having a diameter somewhat larger than a diameter of the projection. In a fitted state, a top surface of the projection of the upper member and a bottom surface of the recess of the lower member need not be completely brought into close contact with each other, and the proper space exists between the projection of the upper member and the recess of the lower member, and the medium ingredient exists therein. In the fitted state, the space surrounded by the top surface of the projection of the upper member, and the bottom surface and the side surface of the recess of the lower member serves as the medium region.

A volume of the medium region can be arbitrarily designed depending on a kind of the analyte to be counted or a scale of examination. For example, the medium region is preferably designed to have a volume of about 1 milliliter to reduce a size of the culture equipment. Moreover, the medium region is preferably designed in such a manner that the medium region is not excessively large (depth of the recess of the lower member is not excessively large) in comparison with an amount of the analyte so that the analyte can be spread wholly to the culture region by pressing or the like, upon putting the upper member on the lower member. Moreover, the medium region is preferably designed in such a manner that, when the upper member has the projection, the medium region is not excessively large (height of the projection of the upper member is not excessively small) in comparison with the amount of the analyte so that the projection can spread out the analyte wholly to the medium region by fitting both.

Meanwhile, if the colonies are vertically stacked, the colonies become hard to be accurately observed and counted. Therefore, the medium region is preferably designed in such a manner that the medium region is not excessively small in comparison with the amount of the analyte (bottom area of the recess in a manner of being not excessively large in a thickness of the medium region).

A thickness of the medium region is preferably adjusted to 0.1 to 1.0 millimeter, for example, but not limited thereto.

The projection of the upper member and the recess of the lower member may have an arbitrary shape as long as a fittable shape is applied. Moreover, the top surface of the projection of the upper member and the bottom surface of the recess of the lower member may be either flat or curved, but from a viewpoint of handleability, a flat surface is preferred.

Then, (c) the medium ingredient is preferably coated onto a portion of the upper member, the portion facing the recess of the lower member upon being put on the lower member, more specifically, at least a part of a portion forming the medium region and/or the recess of the lower member. In an aspect in which the upper member has the projection, (c) the medium ingredient is preferably coated onto at least a part of the projection of the upper member and/or the recess of the lower member. The coated site is ordinarily a portion facing the medium region when the upper member is fitted to the lower member, and is preferably at least a part of the top surface of the projection of the upper member and/or the bottom surface of the recess of the lower member, and is further preferably a whole thereof. In a preferred aspect of the invention, the medium ingredient is coated wholly onto the bottom surface of the recess of the lower member.

According to the invention, a material of the upper member and the lower member is not particularly limited, and a polyacrylic, polyvinyl-based, polyethylene-based or polyester-based polymer or the like can be adopted. Moreover, rigidity of the material does not matter in particular, but when the upper member has no projection, the rigidity at a moderately deformable level is preferred so as to facilitate pressing after addition of a liquid sample.

According to the invention, the upper member and/or the lower member is preferably transparent, and the upper member and the lower member are further preferably transparent. Thus, colonies of microorganisms to be counted can be easily observed and counted from outside without disassembling the culture equipment.

In addition, transparency herein may be at a degree at which an opposite side of the member can be visually observed through the member, and more specifically, visible light transmittance is preferably 70% or more, but not limited thereto.

In the culture equipment according to the invention, the upper member and the lower member may be separated, discretely, or may be united.

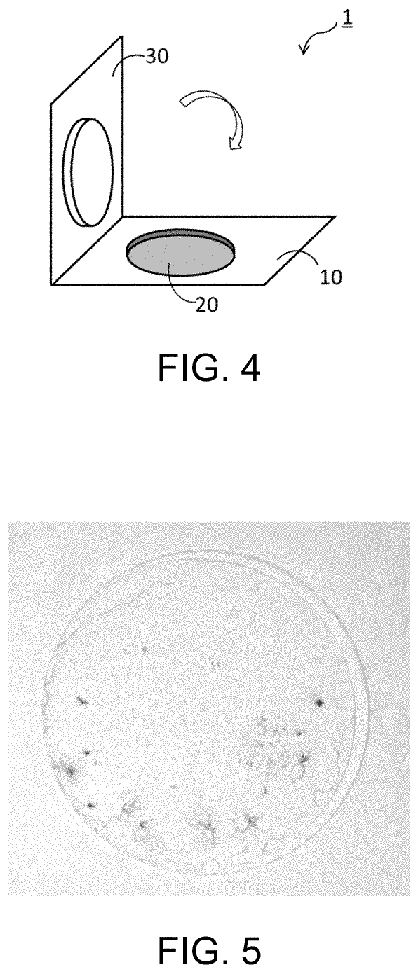

A part of the upper member and a part of the lower member may be continuous by sharing one side as shown in FIG. 4, for example. When the culture equipment has such an aspect, the culture equipment can be used preferably by fitting the projection of the upper member to the recess of the lower member by folding the culture equipment, stacking the upper member with the lower member and putting the upper member on the lower member.

Moreover, in the culture equipment according to the invention, the upper member and the lower member may have a plurality of projections and recesses, respectively. More specifically, the culture equipment may have an aspect in which a plurality of medium regions are formed when in use, which is suitable for treating a plurality of analytes in parallel at one time.

Then, (c) the medium ingredient in the culture equipment according to the invention contains (c1) the polymer compound that can form non-flowable transparent gel without passing through dissolution by heating and without depending on cooling, and (c2) the nutritional ingredient.

According to the invention, (c) the medium ingredient is a material for preparing the medium for culturing the microorganisms. The preparation is ordinarily performed by adding, to the medium ingredient, a liquid analyte containing the microorganisms to be counted, and permeating the analyte into the medium ingredient, as a solvent of gel directly composing the medium.

Here, (c1) the polymer compound that can form non-flowable transparent gel without passing through dissolution by heating and without depending on cooling plays a role of a gelling agent composing the medium.

As (c1) the polymer compound, a material that can be hydrated in an amount preferably 10 times or more, further preferably 20 times or more, and still further preferably 30 times or more its own weight is suitable. The gel suitable for preparing the medium can be formed by such hydration.

The gel formed has no flowability, and therefore the number of existing microorganisms can be accurately counted. Moreover, the gel preferably causes no syneresis. If syneresis is caused, although existence of colonies of the microorganisms can be qualitatively detected, the number of existing microorganisms becomes hard to be accurately counted in several cases. Here, “syneresis” means that water hydrated in the gel is separated from the gel. Moreover, an expression “causing no syneresis” specifically means that water separated from the gel after being left to stand at room temperature for 60 minutes is preferably 0.5% or less, further preferably 0.1% or less of an initial amount of hydrate, for example.

Moreover, the gel formed is transparent. Thus, the colonies of the microorganisms can be accurately counted from outside without disassembling the culture equipment. In addition, “transparency” herein means that, when the polymer compound is added to distilled water at a concentration at which the gel formed does not flow, the visible light transmittance measured by a spectrophotometer (optical path length: 1 cm) is preferably 70% or more (visible light transmittance of distilled water is taken as 100%), but not limited thereto.

Moreover, the polymer compound can form the gel without passing through dissolution by heating and without depending on cooling. Therefore, the operation is simplified and growth of target microorganisms is not hindered. In addition, “heating” herein means raising temperature from room temperature, and specifically means raising temperature to a level at which the microorganisms become inviable, for example, to a temperature over 60° C. Moreover, “cooling” herein means decreasing temperature from a level upon dissolving the polymer compound into the liquid analyte. Moreover, “room temperature” herein ordinarily means 1 to 40° C., preferably 1 to 30° C., and further preferably 20 to 30° C.

Specific examples of such a polymer compound preferably include a material having acrylic acid as a monomer unit, and as long as the material has acrylic acid as the monomer unit, the material is not limited to a homopolymer, and may be a copolymer or a crosslinked polymer.

Specifically, at least one selected from polyacrylic acid and/or a salt thereof and a derivative thereof (hereinafter, also described as “polyacrylic acids”) is preferred.

The gel formed by polyacrylic acids has no flowability, and is hard to cause syneresis, and therefore the number of existing microorganisms can be accurately counted.

Moreover, the gel formed is transparent. Thus, the colonies of the microorganisms can be accurately detected from outside without disassembling the culture equipment.

Moreover, polyacrylic acids can form the gel without passing through dissolution by heating and without depending on cooling, and therefore the operation of forming the medium is simple, the growth of the target microorganisms is not hindered.

As polyacrylic acids, in view of less expensiveness, easy availability and simplicity of gel formation, sodium polyacrylate is particularly preferred.

The gelling agent such as agar, carragheenan and locust bean gum is generally used for the medium for microorganisms, or the like, but the agents described above require heating upon homogeneously solidifying a liquid analyte, and therefore are unsuitable for directly solidifying the liquid analyte containing the microorganisms, or a form of simple culture equipment. Moreover, the gel prepared by solidification using the gelling agent described above is also unsuitable therefor in view of low transparency.

Moreover, polyvinyl alcohol is hard to be homogeneously mixed with the liquid analyte, and also has a problem of easily causing syneresis. Moreover, xanthane gum is also hard to be homogeneously mixed with the liquid analyte to easily form lumps, in which gel solidified also easily becomes opaque.

Carboxymethyl cellulose is unable to solidify the liquid analyte to form flowable gel, and therefore is unsuitable for quantitative detection of the microorganisms.

As polyacrylic acids according to the invention, from a viewpoint of solidifying capability, a material having a degree of polymerization of 10,000 or more is preferred, and a material having a degree of polymerization of 22,000 or more is further preferred. Moreover, the material may be crosslinked or need not be crosslinked.

A concentration of polyacrylic acids when in use according to the invention is not particularly limited, but from a viewpoint of the solidifying capability, is preferably 0.001 g/mL to 0.1 g/mL, and further preferably 0.005 g/mL to 0.05 g/mL. Therefore, the medium ingredient is preferably coated thereon so as to satisfy the range described above in the concentration when in use according to analyte capacity targeted by the culture equipment.

Then, (c2) the nutritional ingredient contained in (c) the medium ingredient is applied for growing the target microorganisms.

The nutritional ingredient is not particularly limited, and specific examples thereof preferably include peptone, an animal meat extract, a yeast extract and a fish meat extract.

In testing methods for drinking water, being one of methods for counting the number of the microorganisms, use of a standard agar medium is recommended, and in testing of water for pharmaceutical use or dialysis water, use of an R2A agar medium is recommended. Therefore, when the medium is used for such an application, a bouillon medium in which agar is excluded from the agar culture media described above, or an ingredient equivalent thereto is preferably incorporated into the medium ingredient according to the invention.

Then, (c) the medium ingredient further preferably contains (c3) a coloration reagent. The reason is that the colonies of the microorganism, the colonies being produced by culture, are easily detected and counted as colored colonies.

Specific examples of the coloration reagent include a redox indicator including 2,3,5-triphenyltetrazolium chloride (TTC) and tetrazolium violet. The indicator described above can be preferably used when all kinds of microorganisms existing in the analyte are desirably counted. When TTC is used, a concentration when in use is preferably 100 mg/L to 1 mg/L, and further preferably 50 mg/L to 10 mg/L.

Moreover, as the coloration reagent, such a material may be used as a substrate (hereinafter, referred to as “enzyme substrate”) to an enzyme owned only by a specific microorganism species, and a compound that can release a pigment compound by being decomposed. The material described above can be preferably used when the specific microorganisms are desirably counted.

Here, the pigment compound may be any of a compound colored under visible light and a fluorescent-colored compound. Specific examples of a functional group that can be released as the colored compound under visible light include a 5-bromo-4-chloro-3-indoxyl group, and 5-bromo-4-chloro-3-indole released is oxidized and fused into 5,5′-dibromo-4,4′-dichloro-indigo, and colored blue. Specific examples of a functional group that can be released as the fluorescent-colored compound include a 4-methyl umbelliferryl group, and 4-methylumbelliferone released emits fluorescence under irradiation with ultraviolet light.

To take the enzyme substrate as an example, when the target microorganisms are Coliform group, 5-bromo-4-chloro-3-indoxlyl-beta-D-galactopyranoside (X-GAL) or 5-bromo-4-chloro-3-indoxyl-beta-D-glucuronic acid can be preferably used, in a case of Staphylococcus aureus, 5-bromo-4-chloro-3-indoxyl phosphate (X-phos) can be preferably used, in a case of Enterococcus, 5-bromo-4-chloro-3-indoxlyl-beta-D-glucopyranoside (X-GLUC) can be preferably used, and in a case of Fungi, X-phos, 5-bromo-4-chloro-3-indoxyl acetate or 5-bromo-4-chloro-3-indoxyl butyrate can be preferably used, respectively. Further, when all of the microorganism species are desirably detected, all of the substances described above may be combined and used.

A concentration of the enzyme substrate when in use is preferably 0.01 to 1.0 g/L, and further preferably 0.2 to 1.0 g/L.

Then, (c) the medium ingredient further contains (c4) a thickening agent. The thickening agent plays a role of an adhesive for stably coating the medium ingredient onto the upper member and/or the lower member.

Such a thickening agent is not particularly limited, as long as the agent does not influence growth of the microorganisms, and specific examples thereof include hydroxypropyl cellulose, polyvinyl pyrrolidone, polyvinyl alcohol, carboxymethyl cellulose or xanthane gum, guar gum, methyl cellulose, starch and a derivative thereof, polyether, hyaluronic acid and collagen.

Then, (c) the medium ingredient arbitrarily may further contain a selective substance, an antibacterial substance, inorganic salts, saccharides, a thickening agent, a pH adjuster or the like, as long as the ingredient does not adversely affect advantageous effects of the invention.

Specific examples of the selective substance include an antibiotic such as polymixin B and vancomycin, and a surfactant such as sodium lauryl sulfate (SDS), Tween 80 and a bile salt such as sodium cholate.

Specific examples of the antibacterial substance include polylysine, protamine sulfate, glycine and sorbic acid.

Specific examples of the inorganic salts include an inorganic acid metal salt such as sodium chloride and sodium thiosulfate, and an organic acid metal salt such as sodium pyruvate, ferric ammonium citrate and sodium citrate.

Specific examples of the saccharides include glucose, lactose, sucrose, xylose, cellobiose and maltose.

Specific examples of the pH adjuster include sodium carbonate and sodium hydrogencarbonate. In addition, from a viewpoint of the growth of the target microorganisms, the composition according to the invention is such a composition to be preferably 6.0 to 8.0, and further preferably 6.5 to 7.5 in the pH when in use.

The culture equipment according to the invention can be produced by an arbitrary method, and one example will be described.

An acrylic plate or the like having a suitable size is used to be applied as an upper member and a lower member. A projection of the upper member and a recess of the lower member only needs to be prepared by adhesion or hollowing of the acrylic plate, pressing using a mold or the like, molding by injection, or the like.

Then, as (c) a medium ingredient, a material prepared by dissolving or a suspending the ingredient into a nonaqueous solvent is partially or wholly applied onto the projection of the upper member and/or the recess of the lower member, and then the resulting material coated. Thus, (c) the medium ingredient can be coated onto culture equipment.

Here, the nonaqueous solvent may be a solvent that can volatilize under ordinary temperature and ordinary pressure, and specific examples thereof preferably include lower alcohol such as ethanol, methanol, propanol and butanol. If the nonaqueous solvent described above is used, the medium ingredient can be coated thereon without gelling (c1) the polymer compound such as polyacrylic acids during production, and therefore the culture equipment can be easily produced.

The culture equipment according to the invention as described above can be preferably used in a method in which the microorganisms in the analyte are cultured, and the number of the microorganisms is counted.

Specifically, the counting method preferably includes a step of adding an analyte to a recess of (b) a lower member; a step of putting (a) an upper member on the recess of (b) the lower member; a step of culturing microorganisms contained in the analyte; and a step of counting the number of colonies of the microorganisms. In the step of putting the upper member on the lower member, the recess is further preferably pressed from outside the equipment. Thus, the analyte added to the recess of the lower member is homogeneously spread out wholly to the medium region. Moreover, (c1) the polymer compound such as polyacrylic acids in the medium ingredient is further quickly gelled by a moisture content in the analyte, and the medium is easily formed.

Moreover, when the culture equipment is used in which the upper member has the projection having a shape that can be mutually fitted to the recess of (b) the lower member through (c) the medium ingredient, the counting method preferably includes a step of adding an analyte to a recess of (b) a lower member of culture equipment; a step of fitting a projection of (a) an upper member to the recess of (b) the lower member; a step of culturing microorganisms contained in the analyte; and a step of counting the number of colonies of the microorganisms.

The analyte added to the recess of the lower member is homogeneously spread out wholly to the medium region by fitting the projection of the upper member to the recess of the lower member. Moreover, (c1) the polymer compound such as polyacrylic acids in the medium ingredient is further quickly gelled by a moisture content of the analyte, and the medium is easily formed.

Culturing conditions of the microorganisms are not particularly limited, and are properly selected according to a kind of the target microorganisms, but are preferably 24 to 48 hours at 35±2° C., for example.

The colonies formed by growth of the target microorganisms emerge in the medium after culture, and the number of the colonies is counted. The number of the colonies of the microorganisms can be counted without disassembling the culture equipment, and only needs to be counted by visually confirming the colonies from outside or analyzing an image picked up by a camera or the like by using image analysis software. According to the counting method of the invention, the number of the colonies can be accurately counted.

The analyte to which the counting method according to the invention can be applied is not particularly limited, and specific examples thereof preferably include a liquid analyte such as drinking water, soft drink, water for industrial use, water for pharmaceutical use, dialysis water and urine. Moreover, the analyte may be a culture solution in which the analyte described above is pre-cultured in tryptic soy broth or the like.

Moreover, the counting method according to the invention can also be applied to a diluted analyte, and even when an amount of the microorganisms in the analyte is 300 CFU/mL or less, for example, such an analyte can be preferably provided for the counting method according to the invention.

### EXAMPLES

Next, the invention will be described in greater detail by way of Examples. The invention is not limited by the Examples.

### (1) Preparation of Culture Equipment

An upper member having a columnar projection having a diameter of 44 mm and a height of 1 mm, and a lower member having a columnar recess having a diameter of 45 mm and a depth of 1 mm in a center of a rectangle having a dimension of 90 mm×60 mm were prepared using a transparent acrylic plate, respectively.

Then, 100 mL in a prepared dose of tryptic soy broth medium powder (Becton, Dickinson and Company), 0.0025 g of TTC, 1 g of sodium polyacrylate (AQUALIC CA; NIPPON SHOKUBAI CO., LTD.) and 0.1 g of hydroxypropyl cellulose were suspended in 50 mL of ethanol. Then, 500 μL of the mixed solution was added to the recess of the lower member made of the acrylic plate prepared as described above, and was homogeneously spread and then the resulting material was dried.

### (2) Sample of a Strain

As a sample strain, Bacillus subtilis NBRC3134 and Escherichia coli NBRC102203 were used. Each sample strain was pre-cultured in a tryptic soy agar medium for 24 hours, and then was suspended into sterile physiological saline using a sterile swab to be a concentration corresponding to McFarland Turbidity Standard No. 1 (about 3.0×10⁸ CFU/mL), and taken as a bacteria stock solution. Then, a bacteria diluted sample having a concentration of several CFU/mL was prepared by repeating 10-fold step dilution of each bacteria stock solution with the sterile physiological saline into a concentration of 10⁻⁸ CFU/mL. Then, a medium was formed by inoculating 1 mL of the bacteria diluted sample into the recess of the lower member of the culture equipment prepared in procedure (1), and immediately fitting the projection of the upper member thereto to homogeneously spread the bacteria diluted sample to the recess, and permeating the bacteria diluted sample into the medium ingredient. The sample was cultured at 35° C. for 24 hours, and then presence or absence of growth was confirmed.

FIG. 5 shows colonies of Bacillus subtilis.

If the culture equipment according to the invention was used, the liquid sample was able to be homogeneously spread wholly to the medium region without depending on a tool such as a spreader or a capillary phenomenon. Moreover, gel of sodium polyacrylate in the medium ingredient was quickly solidified, and after culture, as shown in FIG. 5, red colonies were able to be visually confirmed in transparent gel, and the number thereof was able to be easily counted. Moreover, the culture equipment according to the invention did not have a complicated configuration, and therefore was able to be easily prepared.

### INDUSTRIAL APPLICABILITY

According to the invention, microorganisms in an analyte can be cultured by simple operation, and the number thereof can be easily counted. Moreover, culture equipment according to the invention does not have a complicated configuration, and therefore can be easily produced, and thus is industrially useful.

### REFERENCE SIGNS LIST

1 Culture equipment

10 Lower member

20 Medium ingredient

30 Upper member

1. Culture equipment of microorganisms, comprising (a) an upper member; (b) a lower member having a recess; and (c) a medium ingredient,
wherein (c) the medium ingredient contains (c1) a polymer compound that can forth non-flowable transparent gel without passing through dissolution by heating and without depending on cooling, and (c2) a nutritional ingredient.

2. The culture equipment according to claim 1, wherein (c1) the polymer compound can be hydrated in an amount 10 times or more its own weight.

3. The culture equipment according to claim 1, wherein the gel causes no syneresis.

4. The culture equipment according to claim 1, wherein (c1) the polymer compound has acrylic acid as a monomer unit.

5. The culture equipment according to claim 4, wherein (c1) the polymer compound is at least one selected from polyacrylic acid and/or a salt thereof and a derivative thereof.

6. The culture equipment according to claim 1, wherein (a) the upper member has a projection having a shape that can be mutually fitted to the recess of (b) the lower member through (c) the medium ingredient.

7. The culture equipment according to claim 6, wherein (c) the medium ingredient is coated onto at least a part of the projection of (a) the upper member and/or the recess of (b) the lower member.

8. The culture equipment according to claim 1, wherein (a) the upper member and/or (b) the lower member is transparent.

9. A method, wherein microorganisms in an analyte are cultured and the number of the microorganisms is counted by using the culture equipment according to claim 1.

10. The method according to claim 9, comprising:
a step of adding an analyte to a recess of (b) a lower member;
a step of fitting a projection of (a) an upper member to the recess of (b) the lower member;
a step of culturing microorganisms contained in the analyte; and
a step of counting the number of colonies of the microorganisms.

11. The method according to claim 9, wherein (a) an upper member of the culture equipment has a projection having a shape that can be mutually fitted to a recess of (b) a lower member through (c) a medium ingredient, comprising:
a step of adding an analyte to the recess of (b) the lower member;
a step of fitting the projection of (a) the upper member to the recess of (b) the lower member;
a step of culturing microorganisms contained in the analyte; and
a step of counting the number of colonies of the microorganisms.
